(19) United States
(12) Patent Application Publication
(10) Pub. No.: US 2014/0371527 A1
(43) Pub. Date: Dec. 18, 2014

(54) ENDOSCOPE APPARATUS AND METHOD FOR OPERATING ENDOSCOPE APPARATUS

(71) Applicant: OLYMPUS CORPORATION, TOKYO (JP)

(72) Inventor: Takayuki SATO, Tokyo (JP)

(73) Assignee: OLYMPUS CORPORATION, TOKYO (JP)

(21) Appl. No.: 14/301,651

(22) Filed: Jun. 11, 2014

(30) Foreign Application Priority Data
Jun. 13, 2013 (JP) 2013-124346

(51) Int. Cl. A61B 1/00; A61B 18/14; A61B 1/018

(52) U.S. Cl. CPC A61B 1/00006; A61B 1/018; A61B 18/1492; A61B 2018/00434
USPC 600/106

(57) ABSTRACT

An endoscope apparatus includes a distance information acquisition section that detects the position of a treatment tool and the position of a specific part, and acquires distance information about a distance between the specific part and the treatment tool based on the detected position of the treatment tool and the detected position of the specific part, a degree-of-closeness acquisition section that acquires the degree of closeness between the specific part and the treatment tool based on the distance information, and a treatment tool control section that controls at least one of an incision-related setting and a bleeding arrest-related setting of the treatment tool based on the degree of closeness.

Japanese Patent Application No. 2013-124346 filed on Jun. 13, 2013, is hereby incorporated by reference in its entirety.

### BACKGROUND

The present invention relates to an endoscope apparatus, a method for operating an endoscope apparatus, and the like.

An operation support system has been known that notifies the user that a treatment tool has approached a safety-critical part (i.e., a part that may lead to serious consequences when damaged) during the operation. For example, JP-A-2009-233240 discloses an operation support system that can notify the operator of the relative positional relationship between a treatment tool and a specific part (e.g., safety-critical part) for which contact with the treatment tool must be prevented during the operation. The operation support system disclosed in JP-A-2009-233240 includes a model generation section that generates a tissue model from tissue image data acquired in advance, a distance calculation section that calculates the distance between the end of the treatment tool and the specific part, and a determination section that determines that the end of the treatment tool is positioned close to the specific part when the distance is equal or less than a given threshold value, and notifies the operator that the treatment tool is positioned close to the specific part based on position data relating to the tissue and the treatment tool during the operation.

It is necessary to improve the safety of an operation by preventing a situation in which the operator unintentionally damages a specific part of tissue. The operation support system disclosed in JP-A-2009-233240 notifies the user that the treatment tool has approached a safety-critical part in order to improve the safety of the procedure (treatment).

### SUMMARY

According to one aspect of the invention, there is provided an endoscope apparatus comprising:

a distance information acquisition section that detects a position of a treatment tool and a position of a specific part, and acquires distance information about a distance between the specific part and the treatment tool based on the detected position of the treatment tool and the detected position of the specific part;

a degree-of-closeness acquisition section that acquires a degree of closeness between the specific part and the treatment tool based on the distance information; and

a treatment tool control section that controls at least one of an incision-related setting and a bleeding arrest-related setting of the treatment tool based on the degree of closeness.

According to another aspect of the invention, there is provided a method for operating an endoscope apparatus comprising:

detecting a position of a treatment tool and a position of a specific part, and acquiring distance information about a distance between the specific part and the treatment tool based on the detected position of the treatment tool and the detected position of the specific part;

acquiring a degree of closeness between the specific part and the treatment tool based on the distance information; and

controlling at least one of an incision-related setting and a bleeding arrest-related setting of the treatment tool based on the degree of closeness.

### BRIEF DESCRIPTION OF THE DRAWINGS

FIG. 1 illustrates a basic configuration example of an endoscope apparatus.

FIG. 2 illustrates a configuration example of an endoscope apparatus according to a first embodiment.

FIG. 3 illustrates an example of a look-up table that links distance information and the degree of closeness.

FIG. 4 illustrates an example in which the setting of a treatment tool is controlled corresponding to the degree of closeness (first embodiment).

FIG. 5 illustrates a configuration example of an endoscope apparatus according to a second embodiment.

FIG. 6 illustrates an example when the setting of a treatment tool is controlled corresponding to the degree of closeness (second embodiment).

FIG. 7 illustrates a configuration example of an endoscope apparatus according to a third embodiment.

FIG. 8 illustrates an example when the setting of a treatment tool is controlled corresponding to the degree of closeness (third embodiment).

FIG. 9 illustrates a configuration example of an endoscope apparatus according to a fourth embodiment.

FIG. 10 illustrates a configuration example of an endoscope apparatus according to a fifth embodiment.

### DESCRIPTION OF EXEMPLARY EMBODIMENTS

According to one embodiment of the invention, there is provided an endoscope apparatus comprising:

a distance information acquisition section that detects a position of a treatment tool and a position of a specific part, and acquires distance information about a distance between the specific part and the treatment tool based on the detected position of the treatment tool and the detected position of the specific part;

a degree-of-closeness acquisition section that acquires a degree of closeness between the specific part and the treatment tool based on the distance information; and

a treatment tool control section that controls at least one of an incision-related setting and a bleeding arrest-related setting of the treatment tool based on the degree of closeness.

According to one embodiment of the invention, the degree of closeness between the specific part and the treatment tool is acquired based on the distance information about the distance between the specific part and the treatment tool, and at least one of the incision-related setting and the bleeding arrest-related setting of the treatment tool is controlled based on the degree of closeness. It is possible to improve the safety of the procedure (treatment) by thus controlling the setting of the treatment tool.

Exemplary embodiments of the invention are described below. Note that the following exemplary embodiments do not in any way limit the scope of the invention laid out in the claims. Note also that all of the elements described below in connection with the following exemplary embodiments should not necessarily be taken as essential elements of the invention.

### 1. Outline

An outline of several embodiments of the invention is described below. When performing an operation using an endoscope apparatus, a part (e.g., blood vessel, nerve, or ureter) that should not be damaged (hereinafter may be referred to as “specific part”) may normally be present in (or adjacent to) the operative site. The user (doctor or operator) performs a procedure while avoiding the specific part, or performs a procedure (e.g., a procedure that arrests bleeding, or incises a blood vessel) on the specific part so that an undesirable injury (e.g., bleeding) does not occur. The safety of the operation is impaired if the user unintentionally damages the specific part.

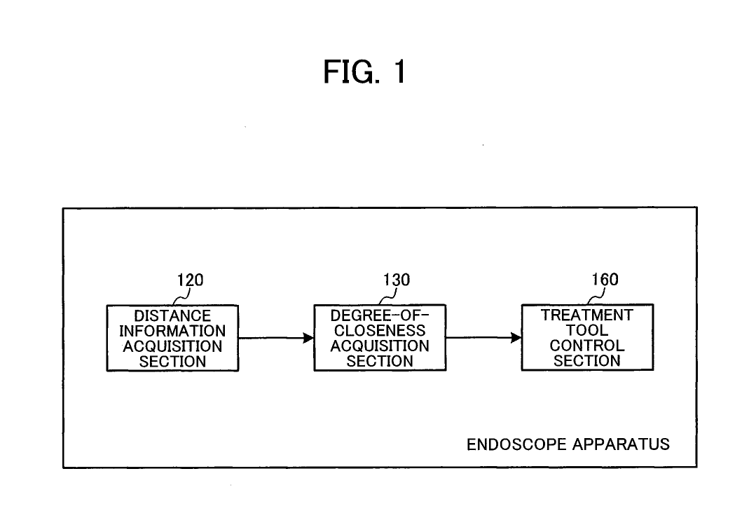

According to several embodiments of the invention, an endoscope apparatus includes a distance information acquisition section 120, a degree-of-closeness acquisition section 130, and a treatment tool control section 160 (see FIG. 1). The distance information acquisition section 120 acquires distance information about the distance between a specific part and a treatment tool (surgical instrument). The degree-of-closeness acquisition section 130 acquires the degree of closeness between the specific part and the treatment tool based on the distance information. The treatment tool control section 160 controls at least one of an incision-related setting and a bleeding arrest-related setting of the treatment tool based on the degree of closeness.

According to this configuration, since the setting (e.g., a setting relating to the incision capability or the bleeding arrest capability) of the treatment tool can be controlled based on the degree of closeness between the specific part (of tissue) and the treatment tool, it is possible to reduce the possibility that the operator unintentionally damages the specific part. The operation support system disclosed in JP-A-2009-233240 is configured to warn the user when the treatment tool has approached the specific part. However, the user may damage the specific part if the setting of the treatment tool is not changed when the treatment tool has approached the specific part. According to several embodiments of the invention, since the setting of the treatment tool can be controlled, safety can be improved as compared with JP-A-2009-233240, for example.

More specifically, the treatment tool control section 160 reduces the incision capability of the treatment tool, or increases the bleeding arrest capability of the treatment tool, when the treatment tool has approached the specific part (e.g., when the degree of closeness has exceeded a threshold value) (as described later with reference to FIG. 4 and the like). For example, when using an electrosurgical knife that utilizes high-frequency electrical energy as the treatment tool, the incision capability (cutting capability) is reduced by reducing the electrical output (electricity) supplied to the electrosurgical knife

According to this configuration, since the incision capability is reduced, or the bleeding arrest capability is increased when the treatment tool has approached the specific part, it is possible to reduce the possibility that the specific part is damaged (or bleeding occurs when the specific part is a blood vessel) even when the user has brought the treatment tool close to the specific part by mistake.

The term “specific part” used herein refers to a safety-critical part (or a part that requires special attention) included in an area that is treated using the treatment tool of the endoscope apparatus. Specifically, an injury to tissue may occur when the user has unintentionally damaged the specific part when performing a procedure (e.g., incision procedure or bleeding arrest procedure) using the treatment tool (e.g., electrosurgical knife or ultrasonic surgical knife). For example, the specific part may be a linear or tubular part such as a blood vessel, a nerve, or a urinary duct.

The term “degree of closeness” used herein refers to an index that indicates the degree of closeness between the treatment tool and the specific part. The degree of closeness increases as the distance between the treatment tool and the specific part decreases. Since the possibility that the specific part is damaged increases as the distance between the treatment tool and the specific part decreases, the degree of closeness is considered to be an index that indicates such a risk. Therefore, the relationship between the distance information and the degree of closeness may be changed corresponding to the risk. For example, when the specific part is a blood vessel, there is a possibility that the blood vessel is damaged, and bleeding occurs. Since the possibility and the amount of bleeding change depending on the incision capability (treatment tool information) of the treatment tool and the attribute (e.g., thickness) (specific part information) of the blood vessel, the degree of closeness may be increased with respect to an identical distance as the incision capability of the treatment tool increases, or the thickness of the blood vessel increases.

The incision-related setting refers to the setting of the treatment tool when incising tissue using the treatment tool. For example, the incision-related setting may be a setting value or a parameter (e.g., the high-frequency output of an electrosurgical knife, or the ultrasonic output of an ultrasonic surgical knife) that determines the incision capability, an incision instruction mode setting (incision mode), or the like. The bleeding arrest-related setting refers to the setting of the treatment tool when arresting bleeding using the treatment tool. For example, the bleeding arrest-related setting may be a setting value or a parameter that determines the bleeding arrest capability, a bleeding arrest instruction mode setting (bleeding arrest mode), or the like.

The distance information is information relating to the distance between the specific part and the treatment tool. The distance information need not necessarily be the distance from the treatment tool to the specific part. Specifically, the distance reference point need not necessarily be the end of the treatment tool, and may be a given position that is set arbitrarily. For example, the distance reference point may be the base of the treatment tool, the end of the imaging section (scope), or the like. Since the treatment tool is inserted into the end of the imaging section, and the shape and the size of the treatment tool are determined in advance, the distance from the reference point can be used instead of the distance from the treatment tool. The end point of the distance is not limited to the position of the specific part that is closest to the treatment tool. For example, when using a knife as the treatment tool, the end point of the distance may be the position of the specific part in the incision direction (along the extension of the tip of the knife). When it is desired to avoid the effect of the treatment tool on the specific part (e.g., a possibility that bleeding occurs when the specific part is a blood vessel), the position of the specific part at which the specific part is affected by the treatment tool may be set to be the end point of the distance.

### 2. First Embodiment

A detailed configuration according to a first embodiment of the invention is described below. An endoscope apparatus according to the first embodiment may be a gastroenterological endoscope apparatus that is inserted into a digestive organ (e.g., an upper gastrointestinal tract (e.g., esophagus or stomach) or a lower gastrointestinal tract (e.g., large intestine)), and used to perform a diagnosis/procedure, or may be a surgical endoscope apparatus that is inserted into an operative site (e.g., brain, abdominal part, or joint) during a surgical operation, and captures the operative site.

Although the first embodiment is decreased below taking an example in which the specific part is a blood vessel, the first embodiment is not limited thereto. Specifically, the specific part may be a safety-critical part (or a part that requires special attention) included in an area that is treated using the treatment tool of the endoscope apparatus.

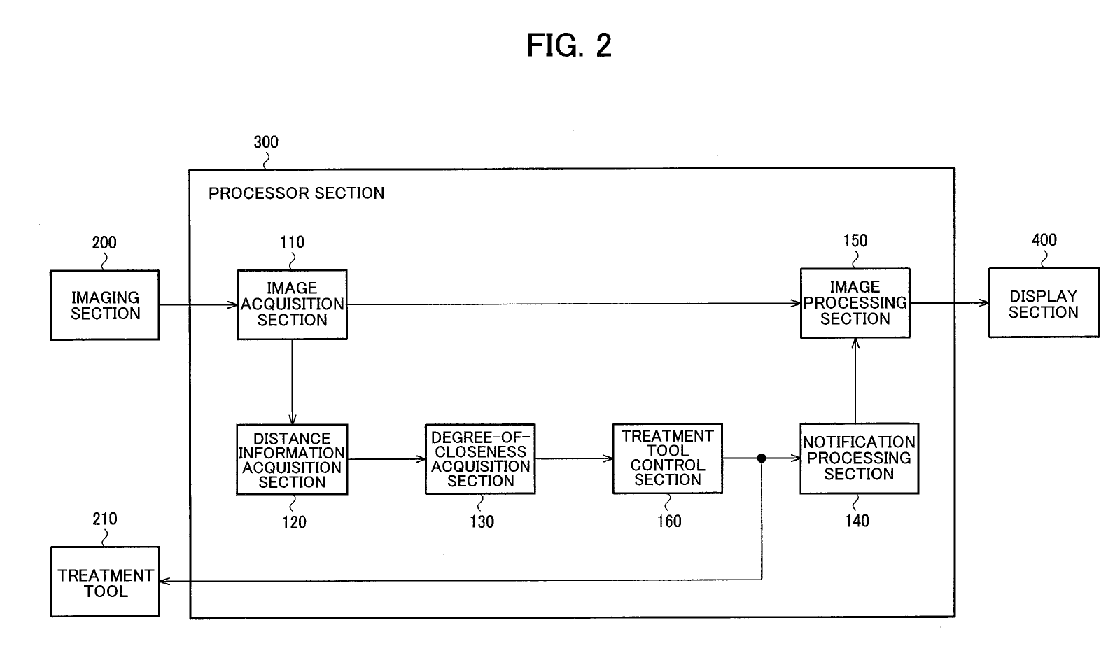

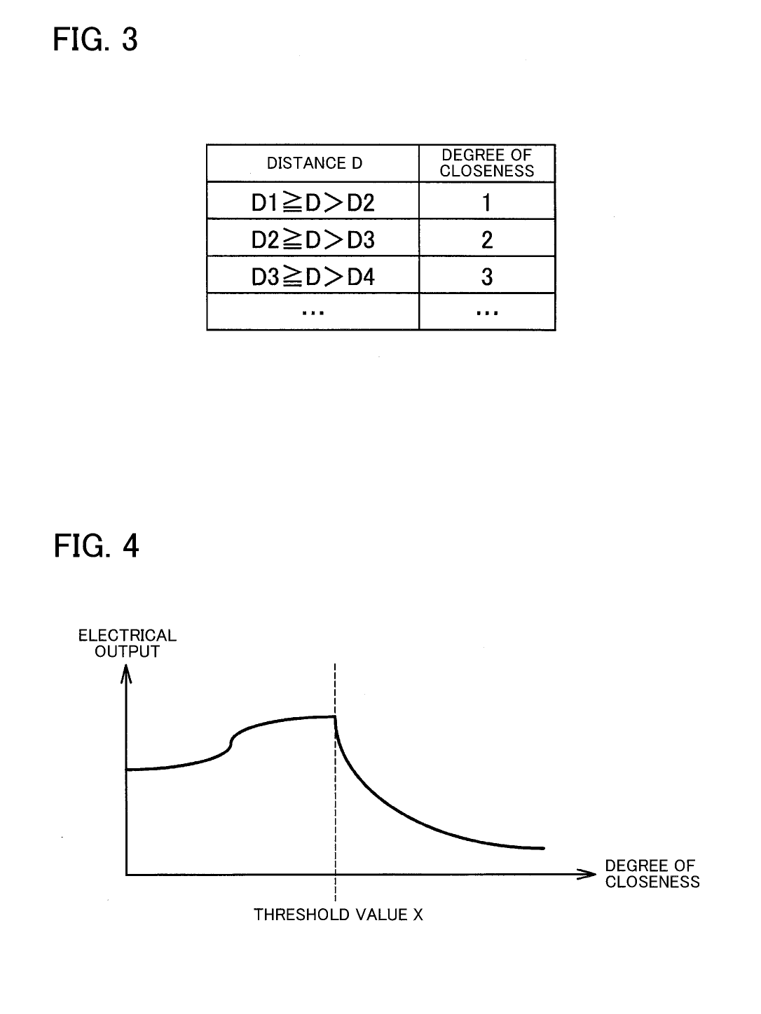

FIG. 2 illustrates a configuration example of the endoscope apparatus according to the first embodiment. The endoscope apparatus includes an imaging section 200 (scope section), a treatment tool 210 (surgical instrument), a processor section 300 (image processor section), and a display section 400.

In the first embodiment, the position of the treatment tool 210 and the position of a blood vessel are detected from the captured image, and the distance information about the distance between the treatment tool 210 and the blood vessel is calculated. The degree of closeness is acquired based on the distance information, and the setting of the treatment tool 210 is controlled based on the degree of closeness.

The imaging section 200 includes an image sensor (e.g., CCD or CMOS sensor). The imaging section 200 captures the observation target (object) using the image sensor, and outputs the captured image (image data) to the processor section 300.

The processor section 300 performs image processing on the captured image, and controls each section of the endoscope apparatus. The processor section 300 includes an image acquisition section 110, a distance information acquisition section 120, a degree-of-closeness acquisition section 130, a notification processing section 140, an image processing section 150, and a treatment tool control section 160.

The image acquisition section 110 receives the captured image (image data) output from the imaging section 200, and outputs the captured image to the image processing section 150 and the distance information acquisition section 120.

The image processing section 150 performs image processing on the captured image, and outputs the resulting image to the display section 400. For example, the image processing section 150 performs a white balance process, a gamma correction process, a highlight process, a scaling process, a distortion correction process, a noise reduction process, and the like.

The distance information acquisition section 120 detects a blood vessel (blood vessel structure) from the captured image, and acquires position information about the blood vessel. The distance information acquisition section 120 detects a treatment tool from the captured image, and acquires position information about the treatment tool. The distance information acquisition section 120 calculates the two-dimensional distance (or the three-dimensional distance described later) between the treatment tool and the blood vessel from the position information about the blood vessel and the position information about the treatment tool. The treatment tool may be detected by utilizing the fact that a treatment tool is normally made of a metal. For example, the captured image is converted into a brightness (luminance) image, and pixels having a brightness value equal to or larger than a given threshold value are detected. The detected pixels are divided into a plurality of groups through contour detection, and a group having a pixel count equal to or larger than a given pixel count is detected as the treatment tool. A blood vessel may be detected based on the difference in brightness from an area in which a blood vessel is not present. The end of the detected pixel group is considered to be the end of the treatment tool, and the distance from the end of the treatment tool to the blood vessel is calculated. Note that the treatment tool detection method and the blood vessel detection method are not limited to the above methods. For example, a treatment tool or a blood vessel may be detected from a color feature using a color difference image (Cr or Cb) or the like.

The degree-of-closeness acquisition section 130 calculates the degree of closeness between the blood vessel and the treatment tool based on the distance information, and outputs the calculated degree of closeness to the treatment tool control section 160. The degree of closeness increases as the distance between the blood vessel and the treatment tool decreases. For example, the degree-of-closeness acquisition section 130 stores a look-up table (see FIG. 28), and converts a two-dimensional distance D into the degree of closeness by referring to the look-up table. Note that the relationship “D1>D2>D3>D4” is satisfied. The degree-of-closeness acquisition section 130 may calculate the degree of closeness using a function that links the two-dimensional distance D to the degree of closeness. For example, the degree of closeness is represented by f(D)=α/D (where a is a given coefficient).

The treatment tool control section 160 changes the setting of the treatment tool 210 corresponding to the degree of closeness. Specifically, the treatment tool 210 has a tissue incision function or a bleeding arrest function, and the incision capability or the bleeding arrest capability is adjusted according to the electrical output (or vibrational output). For example, the treatment tool control section 160 changes the electrical output (or vibrational output) setting value with respect to the degree of closeness, and reduces the incision capability when the degree of closeness is high (see FIG. 4). Specifically, the user can arbitrarily set the incision capability when the degree of closeness is smaller than a threshold value X, and the treatment tool control section 160 gradually reduces the incision capability as the degree of closeness increases when the degree of closeness is larger than the threshold value X.

The treatment tool 210 may be a treatment tool that generates high-frequency energy as the electrical output (e.g., electrosurgical knife or bipolar device), or may be a treatment tool that generates ultrasonic vibrational energy as the vibrational output (e.g., ultrasonic surgical knife). These treatment tools are configured to overheat tissue using high-frequency energy or vibrational energy, and denature the tissue due to heat to implement incision or arrest bleeding. For example, an incision tool outputs high energy sufficient to destroy tissue through denaturation. A bleeding arrest tool outputs relatively low energy that coagulates tissue through denaturation. When using a treatment tool that is configured so that the mode can be switched between an incision mode and a bleeding arrest mode, the mode is switched between the incision mode and the bleeding arrest mode by switching the output energy.

Note that the treatment tool 210 need not necessarily be controlled using the configuration illustrated in FIG. 4. It suffices that the treatment tool 210 be controlled so that the possibility that the specific part is damaged is reduced when the treatment tool 210 has approached the specific part. For example, the bleeding arrest capability may be increased to prevent unintentional bleeding when the degree of closeness has exceeded the threshold value X. Alternatively, the mode may be switched from the incision mode to the bleeding arrest mode when the degree of closeness has exceeded the threshold value X. When the specific part is a part (e.g., nerve) for which it is indispensable to avoid damage, the output may be set to 0 (i.e., may be set to a level equal to or lower than a given level in a broad sense) when the degree of closeness has exceeded the threshold value X.

The notification processing section 140 notifies the user of the control state of the treatment tool 210 based on the setting (setting value or mode setting) controlled by the treatment tool control section 160. The control state may be the incision capability reduction state or the mode setting (e.g., incision mode), for example. The notification processing section 140 outputs a display setting that indicates the control state to the image processing section 150, and the image processing section 150 superimposes the display setting on the captured image, and outputs the captured image to the display section 400 as display data. The display section 400 displays the control state of the treatment tool together with the diagnostic image.

Note that the control state of the treatment tool need not necessarily be displayed as an image. The user may be notified of the control state of the treatment tool using sound, vibrations, or light emitted from an LED, for example.

### 3. Modifications of first embodiment

Note that various modifications and variations may be made of the first embodiment. Various modifications of the first embodiment are described below.

Although the first embodiment has been described above taking an example in which the two-dimensional distance is acquired as the distance information, the three-dimensional distance may be acquired as the distance information (first modification).

According to the first modification, the imaging section 200 includes a stereo optical system that can capture a stereo image. The stereo optical system includes two imaging optical systems that are disposed to have a parallax, for example. The distance information acquisition section 120 performs a matching process on a stereo image having a parallax to calculate the distance in the depth direction at each position of the image. The distance information acquisition section 120 detects the three-dimensional position of the treatment tool and the three-dimensional position of the blood vessel based on the two-dimensional position of the treatment tool, the two-dimensional position of the blood vessel, and the distance in the depth direction, and calculates the three-dimensional distance between the treatment tool and the blood vessel. The degree-of-closeness acquisition section 130 converts the three-dimensional distance into the degree of closeness is the same manner as described above in connection with the first embodiment.

Note that the distance in the depth direction may be detected using a method other than the stereo imaging method. For example, the imaging section 200 may include an autofocus optical system, and the distance to the treatment tool or the blood vessel in the depth direction may be estimated from the lens position when the treatment tool or the blood vessel has been brought into focus through an autofocus process. Alternatively, the distance to the treatment tool or the blood vessel in the depth direction may be estimated from the brightness of the image by utilizing the fact that the brightness of illumination light applied from the end of the imaging section 200 decreases as the distance to the object increases.

Although the first embodiment has been described above taking an example in which the position of the treatment tool is detected from the image captured by the imaging section 200, the position of the treatment tool may be detected based on the output from a position sensor (second modification).

According to the second modification, the position sensor detects the position of the treatment tool, and outputs the position of the treatment tool to the distance information acquisition section 120. The distance information acquisition section 120 acquires the distance information about the distance from the treatment tool to the blood vessel based on the position of the treatment tool and the position of the blood vessel detected from the image. For example, a magnetic coil is incorporated in the treatment tool 210, and the position sensor detects the position of the treatment tool by sensing magnetism generated from the magnetic coil.

Although the first embodiment has been described above taking an example in which the position information about a blood vessel (i.e., the specific part of tissue) is acquired, position information about a nerve, a urine tube, or the like may also be acquired as long as the structure thereof can be detected (third modification).

According to the third modification, the structure of tissue is determined using an image by administering indocyanine green (ICG) (i.e., near infrared fluorescence probe), and observing fluorescence emitted from ICG that circulates inside the body, for example. This makes it possible to determine the structure of a urine tube through image recognition, for example. The structure of a nerve may be determined using an image by administering a specific fluorescent agent or the like, for example.

Although the first embodiment has been described above taking an example in which the position of the end of the treatment tool 210 is detected from the image, a marker may be provided to the treatment tool 210, and the position of the surgical instrument may be acquired through image recognition (fourth modification).

According to the fourth modification, a marker is provided to the end of the treatment tool 210, and the distance information acquisition section 120 detects the marker to detect the position of the treatment tool 210. Alternatively, the marker may be provided to a part (e.g., the grip of a knife) of the treatment tool 210 other than the end thereof. In this case, the distance information acquisition section 120 acquires the distance from the marker to the end of the knife based on physical information (e.g., length and width) about the treatment tool 210, and estimates the position of the end of the knife from the acquired distance.

According to the first embodiment, the treatment tool control section 160 reduces the incision capability of the treatment tool 210 when the degree of closeness is larger than the threshold value.

According to this configuration, since the incision capability is reduced when the distance between the treatment tool 210 and the specific part has become equal to or less than a given value, it is possible to improve the safety of the operation by preventing a situation in which the user damages the specific part included in the operative site by mistake.

The term “incision capability” used herein refers to information that indicates the capability of the treatment tool 210 to incise tissue. For example, the term “incision capability” used herein refers to information that indicates the range (e.g., length or depth) in which tissue is incised when an electrical output (or vibrational output) is output from the treatment tool 210 in response to an instruction from the user, or under control of the treatment tool control section 160.

According to the first embodiment, the notification processing section 140 notifies the user of the control state implemented by the treatment tool control section 160. More specifically, when the treatment tool control section 160 reduces the incision capability of the treatment tool 210 when the degree of closeness is larger than the threshold value, the notification processing section 140 notifies the user that the incision capability is reduced.

According to this configuration, since the user can determine the control state of the treatment tool 210, the user can perform an appropriate operation corresponding to the control state. Specifically, since the endoscope apparatus draws the user's attention by notifying the control state, and a situation in which the control state is changed before the user notices does not occur, it is possible to ensure the safety of the procedure.

According to the method disclosed in JP-A-2009-233240, it is necessary to acquire tissue image data in advance using MRI or the like, and a position detection device is required to detect treatment tool position data when performing an operation. Therefore, the method disclosed in JP-A-2009-233240 has a problem in that a large-scale apparatus is required.

According to the first embodiment, the image acquisition section 110 acquires the captured image captured by the imaging section 200, the captured image including an image of the specific part and the treatment tool 210. The distance information acquisition section 120 detects the position of the treatment tool 210 and the position of the specific part from the captured image, and acquires the distance information about the distance from the treatment tool 210 to the specific part based on the detected position of the treatment tool 210 and the detected position of the specific part.

According to this configuration, since it is possible to detect a situation in which the treatment tool 210 is approaching the specific part in real time from the captured image, and notify the user of the situation as the degree of closeness, it is unnecessary to acquire image data relating to the specific part in advance. Since it is unnecessary to provide a device for acquiring image data relating to the specific part in advance, and a device or a sensor (e.g., ultrasonic imaging device, MRI, or GPS transmitter-receiver) that detects the position of the treatment tool 210 without using the captured image, it is possible to implement a compact apparatus.

For example, when detecting the treatment tool 210 from the image, and performing a procedure using a knife, the end of the knife may be hidden behind tissue, and may not be captured. For example, the end of the knife may be hidden behind tissue when incising tissue using the knife, and it may be impossible to determine the end of the knife from the image.

However, when the marker is provided to the grip of the treatment tool 210, for example, the position of the end of the treatment tool 210 can be estimated even when the end of the knife is hidden behind tissue, and an accurate degree of closeness can be calculated.

### 4. Second Embodiment

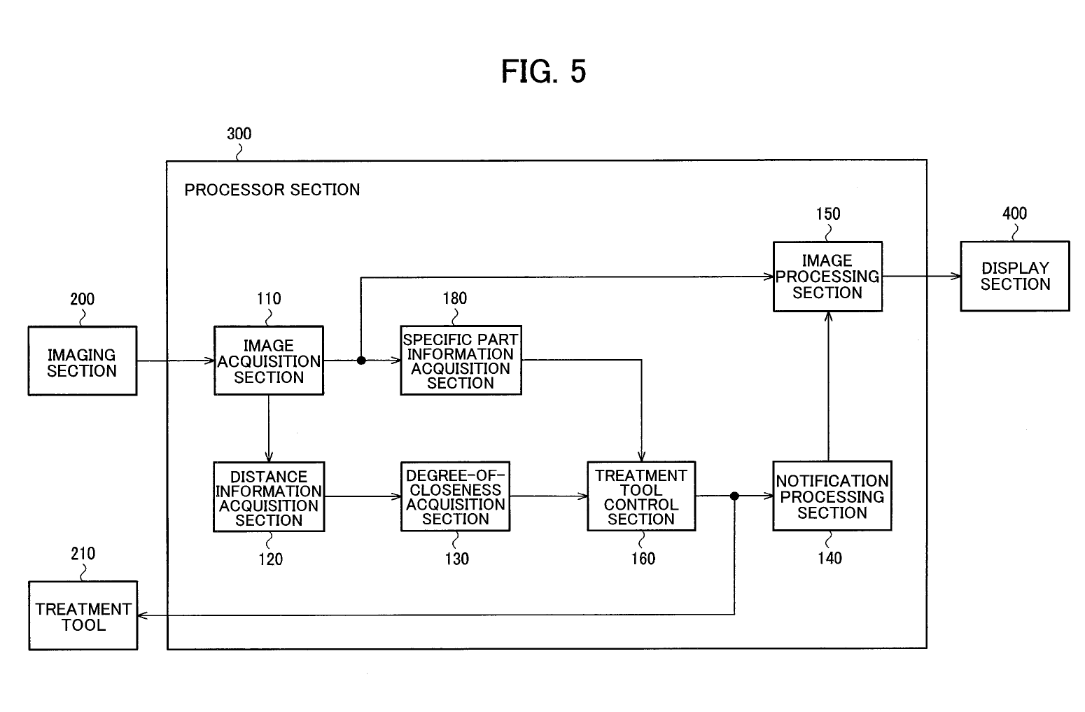

FIG. 5 illustrates a configuration example of an endoscope apparatus according to a second embodiment of the invention. The endoscope apparatus includes an imaging section 200, a treatment tool 210, a processor section 300, and a display section 400. The processor section 300 includes an image acquisition section 110, a distance information acquisition section 120, a degree-of-closeness acquisition section 130, a notification processing section 140, an image processing section 150, a treatment tool control section 160, and a specific part information acquisition section 180. Note that the same elements as those described above in connection with the first embodiment are respectively indicated by the same reference signs, and description thereof is appropriately omitted.

In the second embodiment, the setting of the treatment tool 210 is controlled based on specific part information that is attribute information about the specific part, and the degree of closeness of the specific part.

Specifically, the specific part information acquisition section 180 detects the boundary of a blood vessel from the captured image acquired by the image acquisition section 110, and acquires the diameter (width) of the blood vessel as the specific part information based on the detection result. Since it is considered that a blood vessel normally has a circular cross-sectional shape, the width of the blood vessel within the image is determined to be the diameter of the blood vessel. Note that the distance information acquisition section 120 and the specific part information acquisition section 180 may or may not independently perform the process that detects a blood vessel from the captured image.

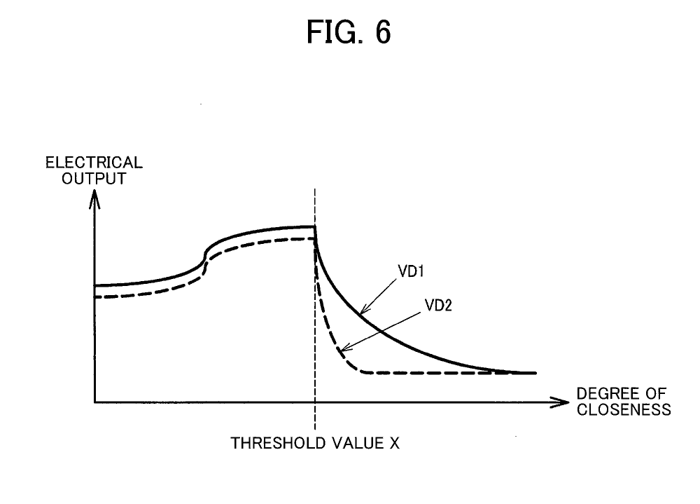

The treatment tool control section 160 controls the setting of the treatment tool 210 based on the diameter of the blood vessel and the degree of closeness. FIG. 6 illustrates a control example. VD1 indicates the relationship between the degree of closeness and the electrical output (or vibrational output) when the diameter of the blood vessel is smaller than a given value, and VD2 indicates the relationship between the degree of closeness and the electrical output (or vibrational output) when the diameter of the blood vessel is larger than the given value. The treatment tool control section 160 reduces the incision capability of the treatment tool 210 when the degree of closeness is larger than a threshold value X. When the diameter of the blood vessel is larger than the given value (VD2), the treatment tool control section 160 determines that bleeding occurs to a large extent when the blood vessel is cut by mistake, and immediately reduces the incision capability of the treatment tool 210. When the diameter of the blood vessel is smaller than the given value (VD1), the treatment tool control section 160 determines that bleeding occurs to only a small extent when the blood vessel is cut by mistake, and does not immediately reduces the incision capability of the treatment tool 210. Specifically, the treatment tool control section 160 more quickly reduces the incision capability of the treatment tool 210 in response to an increase in the degree of closeness as the diameter of the blood vessel increases.

According to the second embodiment, the specific part information acquisition section 180 acquires the specific part information that is the attribute information about the specific part. The treatment tool control section 160 controls at least one of the incision-related setting and the bleeding arrest-related setting of the treatment tool based on the degree of closeness and the specific part information.

This makes it possible to appropriately control the treatment tool 210 corresponding to the attribute of the specific part. Specifically, it is possible to change the setting of the treatment tool 210 with respect to the degree of closeness corresponding to the attribute of the specific part.

Although the second embodiment has been described above taking an example in which the diameter (thickness) of the blood vessel is acquired as the specific part information, the second embodiment is not limited thereto. It suffices that the attribute information about the specific part be information corresponding to the attribute of the specific part present in the procedure target area. For example, when the specific part is a blood vessel, the attribute information may be information that indicates the type (e.g., artery, vein, or peripheral blood vessel) of the blood vessel, the thickness of the blood vessel, the part (e.g., lower gastrointestinal tract or upper gastrointestinal tract) where the blood vessel is present, the tissue (e.g., mucous membrane, fat, or muscle) where the blood vessel is present, or the like.

In the second embodiment, the specific part information may be thickness information about the specific part. The treatment tool control section 160 may reduce the incision capability of the treatment tool 210 when the degree of closeness is larger than the threshold value X, and increase the degree of reduction in the incision capability as the thickness of the specific part increases. Specifically, the specific part may be a blood vessel, and the thickness information may be the diameter of the blood vessel.

This makes it possible to optimally control at least one of the incision-related setting and the bleeding arrest-related setting corresponding to the thickness of the (adjacent) blood vessel. Specifically, since bleeding occurs to a large extent when a blood vessel having a large thickness is damaged, safety can be improved by quickly reducing the incision capability of the treatment tool 210 when the blood vessel has a large thickness. It is possible to prevent a situation in which the treatment time unnecessarily increases by decreasing the degree of reduction in the incision capability when the blood vessel has a small thickness.

The term “incision capability” used herein refers to information that indicates the capability of the treatment tool 210 to incise tissue. For example, the term “incision capability” used herein refers to information that indicates the range (e.g., length or depth) in which tissue is incised when an electric current is caused to flow through the knife (or when a voltage is applied to the knife) according to an instruction issued by the user.

In the second embodiment, the specific part information may be information that indicates the type of the specific part. The treatment tool control section 160 may reduce the incision capability as the degree of closeness increases when the degree of closeness is larger than the threshold value X provided that the specific part is a blood vessel, and set the incision capability to be equal to or less than a given level when the degree of closeness is larger than the threshold value X provided that the specific part is a nerve.

This makes it possible to optimally control at least one of the incision-related setting and the bleeding arrest-related setting corresponding to the type of the (adjacent) specific part. Specifically, the risk of damage to the specific part can be minimized by setting the incision capability to be equal to or less than a given level (e.g., 0) when the specific part is a nerve or the like for which it is indispensable to avoid damage.

### 5. Third Embodiment

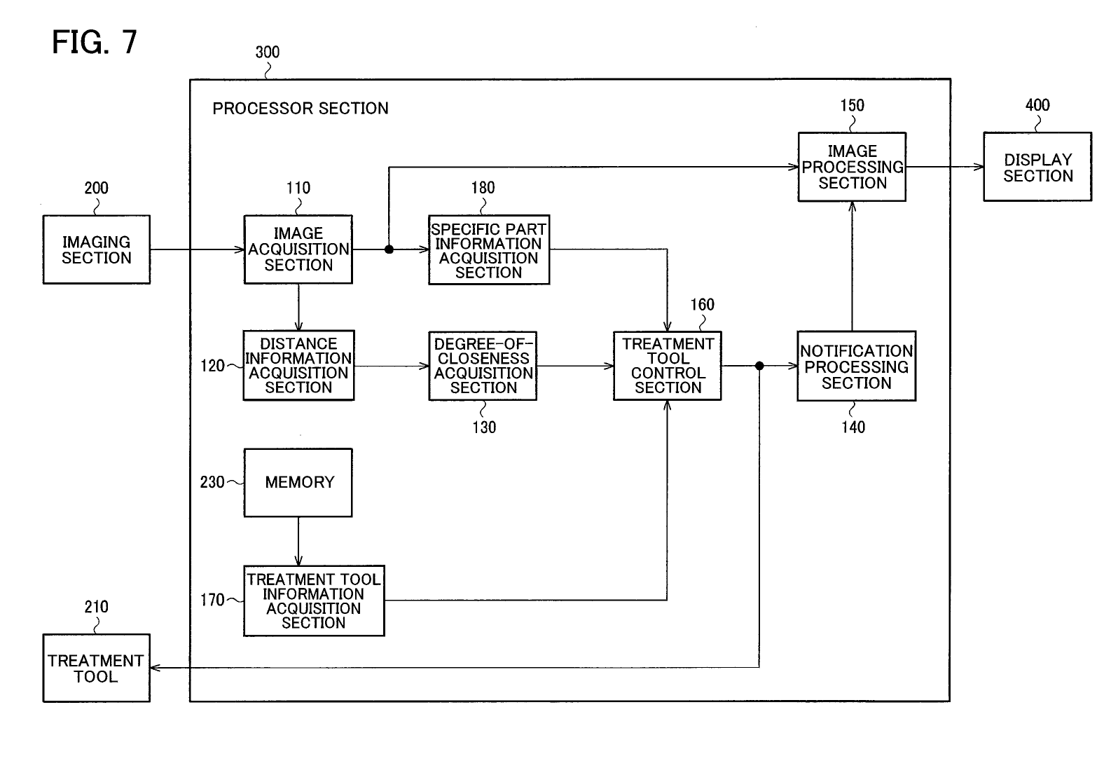

FIG. 7 illustrates a configuration example of an endoscope apparatus according to a third embodiment of the invention. The endoscope apparatus includes an imaging section 200, a treatment tool 210, a processor section 300, and a display section 400. The processor section 300 includes an image acquisition section 110, a distance information acquisition section 120, a degree-of-closeness acquisition section 130, a notification processing section 140, an image processing section 150, a treatment tool control section 160, a treatment tool information acquisition section 170, a specific part information acquisition section 180, and a memory 230. Note that the same elements as those described above in connection with the above embodiments are respectively indicated by the same reference signs, and description thereof is appropriately omitted.

In the third embodiment, the setting of the treatment tool 210 is controlled based on treatment tool information that is attribute information about the treatment tool 210, and the degree of closeness between the treatment tool 210 and the specific part.

The memory 230 stores a model number parameter (ID information) of the treatment tool 210. The memory 230 may store information about the application, the applied part, the size, the shape, and the like of the treatment tool 210.

The treatment tool information acquisition section 170 acquires the bleeding arrest capability of the treatment tool 210 as the treatment tool information based on the information read from the memory 230. For example, the treatment tool information acquisition section 170 stores a look-up table that links the information read from the memory 230 with the incision capability, and acquires the bleeding arrest capability by referring to the look-up table. The treatment tool information acquisition section 170 may acquire the bleeding arrest capability by a function using the information read from the memory 230 as an argument.

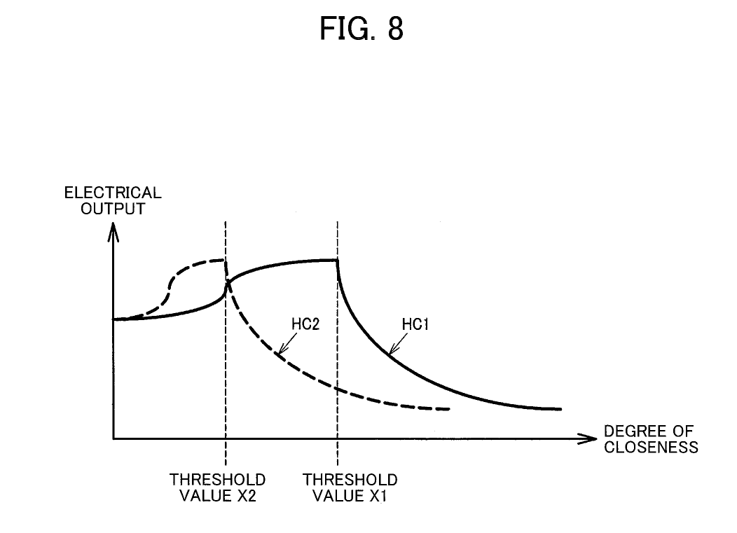

The treatment tool control section 160 controls the setting of the treatment tool 210 based on the bleeding arrest capability and the degree of closeness. FIG. 8 illustrates a control example. HC1 indicates the relationship between the degree of closeness and the electrical output (or vibrational output) when the bleeding arrest capability is high, and HC2 indicates the relationship between the degree of closeness and the electrical output (or vibrational output) when the bleeding arrest capability is low. When the bleeding arrest capability of the treatment tool 210 is high (HC1), the treatment tool control section 160 reduces the incision capability of the treatment tool 210 when the degree of closeness is larger than a first threshold value X1. When the bleeding arrest capability of the treatment tool 210 is low (HC2), the treatment tool control section 160 reduces the incision capability of the treatment tool 210 when the degree of closeness is larger than a second threshold value X2 (X2<X1). For example, an ultrasonic surgical knife has a high incision capability as compared with an electrosurgical knife (high-frequency knife), but has a low bleeding arrest capability as compared with an electrosurgical knife, and an electrosurgical knife has a low incision capability as compared with an ultrasonic surgical knife, but has a high bleeding arrest capability as compared with an ultrasonic surgical knife. Specifically, the threshold value when using an electrosurgical knife is set to be larger than the threshold value when using an ultrasonic surgical knife

### 6. Modifications of third embodiment

Note that various modifications and variations may be made of the third embodiment. Various modifications of the third embodiment are described below.

Although the third embodiment has been described above taking an example in which the control process that controls the treatment tool 210 based on the diameter of the blood vessel and the control process that controls the treatment tool 210 based on the bleeding arrest capability are performed independently, the treatment tool 210 may be controlled by combining these control processes. For example, when using a treatment tool 210 having a high bleeding arrest capability, the incision capability may not be reduced even when the diameter of the blood vessel is larger than the threshold value X.

Although the third embodiment has been described above taking an example in which the bleeding arrest capability is acquired based on the information read from the memory 230, the bleeding arrest capability may be acquired based on setting information from the user. For example, when the treatment tool 210 is configured so that the bleeding arrest capability can be adjusted (switched), the bleeding arrest capability that has been adjusted (switched) by the user may be acquired as the treatment tool information.

Although the third embodiment has been described above taking an example in which the memory 230 is provided in the processor section 300. the memory 230 may be provided in the treatment tool 210 or the imaging section 200. In this case, the treatment tool information acquisition section 170 reads the parameter of the treatment tool 210 from the memory 230 provided in the treatment tool 210 or the imaging section 200.

According to the third embodiment, the treatment tool information acquisition section 170 acquires the treatment tool information that is the attribute information about the treatment tool 210. The treatment tool control section 160 controls at least one of the incision-related setting and the bleeding arrest-related setting of the treatment tool 210 based on the degree of closeness and the treatment tool information.

This makes it possible to appropriately control the treatment tool 210 corresponding to the attribute of the treatment tool 210. Specifically, it is possible to change the setting of the treatment tool 210 with respect to the degree of closeness corresponding to the attribute of the treatment tool 210.

Although the third embodiment has been described above taking an example in which the treatment tool information is the bleeding arrest capability, the third embodiment is not limited thereto. It suffices that the attribute information about the treatment tool 210 be information corresponding to the attribute of the treatment tool 210. For example, when the treatment tool 210 is a knife, information corresponding to the incision capability of the knife may be a current that is caused to flow through the knife, a voltage applied to the knife, the material of the knife, the shape of the blade, the size of the blade, the procedure mode (e.g., bleeding arrest mode or incision mode) set to the knife, or the like. The attribute information about the treatment tool 210 may be information about a part (e.g., lower gastrointestinal tract or upper gastrointestinal tract) to which the treatment tool 210 is applied, ID information linked to the treatment tool 210, or the like.

According to the third embodiment, the treatment tool control section 160 reduces the incision capability of the treatment tool 210 when the degree of closeness is larger than the threshold value (X1 or X2), and changes the threshold value corresponding to the treatment tool information. More specifically, the specific part is a blood vessel, and the treatment tool information is the bleeding arrest capability of the treatment tool 210. The treatment tool control section 160 decreases the threshold value (X1>X2) as the bleeding arrest capability decreases.

This makes it possible to optimally control at least one of the incision-related setting and the bleeding arrest-related setting corresponding to the bleeding arrest capability. Specifically, since it may take time to arrest bleeding when the bleeding arrest capability is low, safety can be improved by reducing the incision capability of the treatment tool 210 even when the treatment tool 210 is situated away from the specific part. When the treatment tool 210 has a high bleeding arrest capability (i.e., bleeding can be arrested within a short time), it is possible to prevent a situation in which the treatment time unnecessarily increases, by reducing the incision capability of the treatment tool 210 when the treatment tool 210 is situated closer to the specific part.

The term “bleeding arrest capability” used herein refers to information that indicates the capability of the treatment tool 210 to arrest bleeding. For example, the treatment tool 210 for arresting bleeding may be a device that arrests bleeding by holding a blood vessel, and heating/coagulating the tissue by applying high-frequency waves or ultrasonic waves. The bleeding arrest capability of such a device the blood vessel coagulation range (e.g., width) or time, the thickness of a blood vessel from which bleeding can be arrested, the bleeding arrest reliability, or the like.

The treatment tool information is information that indicates the type of the treatment tool 210. The treatment tool control section 160 sets the threshold value to the first threshold value X1 when the treatment tool 210 is an electrosurgical knife, and sets the threshold value to the second threshold value X2 that is smaller than the first threshold value X1 when the treatment tool 210 is an ultrasonic surgical knife.

This makes it possible to optimally control at least one of the incision-related setting and the bleeding arrest-related setting corresponding to the type of the treatment tool 210. Specifically, the risk of damage to the specific part can be minimized by reducing the incision capability of the treatment tool 210 even when the treatment tool 210 is situated away from the specific part when the treatment tool 210 is an ultrasonic surgical knife having a relatively low bleeding arrest capability.

### 7. Fourth Embodiment

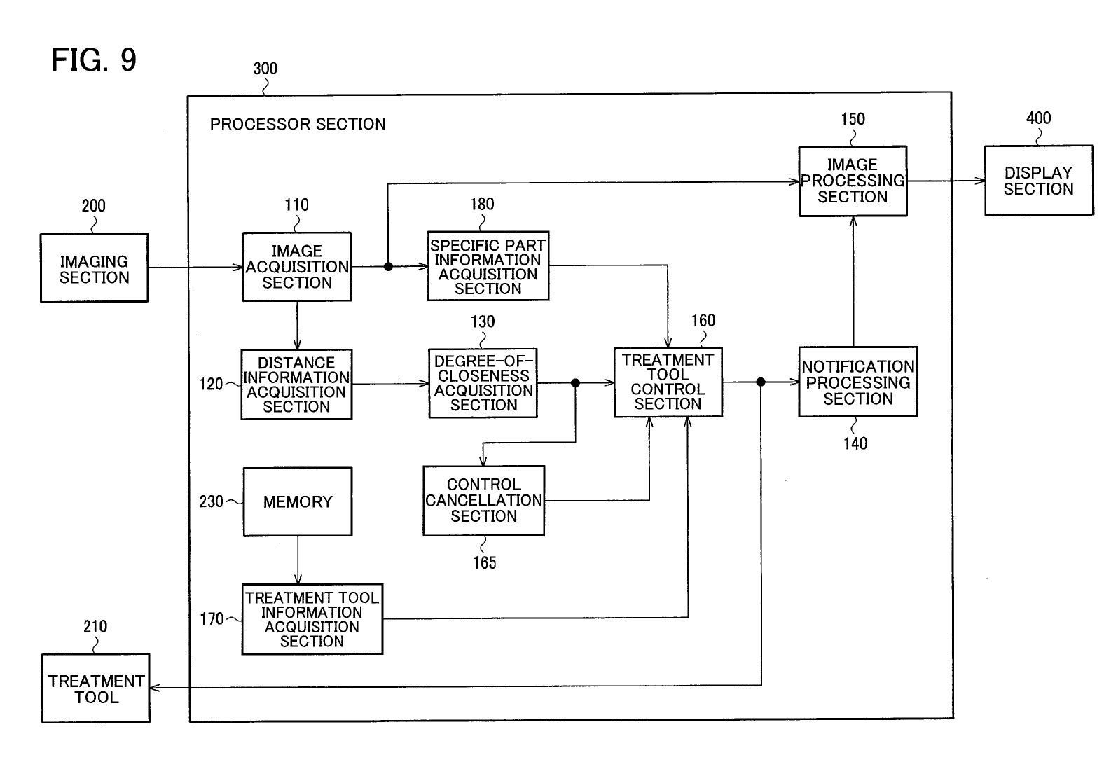

FIG. 9 illustrates a configuration example of an endoscope apparatus according to a fourth embodiment of the invention. The endoscope apparatus includes an imaging section 200, a treatment tool 210, a processor section 300, and a display section 400. The processor section 300 includes an image acquisition section 110, a distance information acquisition section 120, a degree-of-closeness acquisition section 130, a notification processing section 140, an image processing section 150, a treatment tool control section 160, a control cancellation section 165, a treatment tool information acquisition section 170, a specific part information acquisition section 180, and a memory 230. Note that the same elements as those described above in connection with the above embodiments are respectively indicated by the same reference signs, and description thereof is appropriately omitted.

In the fourth embodiment, the control process performed on the treatment tool 210 corresponding to the degree of closeness is canceled when a given condition has been satisfied.

More specifically, the control cancellation section 165 cancels the control process performed by the treatment tool control section 160 based on at least one of an input from the user and the elapsed time. The treatment tool control section 160 reduces the incision capability of the treatment tool 210 when the degree of closeness is larger than the threshold value. The control cancellation section 165 cancels the control process that reduces the incision capability of the treatment tool 210 when the user has performed an input, or when the degree of closeness has not changed by a given amount within a given time, or when the user has performed an input, and the degree of closeness has not changed by the given amount within the given time.

Specifically, the control cancellation section 165 starts a timer provided therein when the degree of closeness has not changed by the given amount, and outputs a cancellation signal to the treatment tool control section 160 when the given time has elapsed. The control cancellation section 165 also outputs the cancellation signal when the operator has issued a cancellation instruction. The treatment tool control section 160 stops reducing the incision capability of the treatment tool 210 upon reception of the cancellation signal from the control cancellation section 165.

The incision capability is reduced when the treatment tool 210 has approached the specific part, and the user becomes unable to incise tissue. In this case, when the user has moved the treatment tool 210 away from the specific part, it is considered that the user does not intend to treat the specific part. When the user does not move the treatment tool 210 even when the incision capability has been reduced, it is considered that the user intends to treat the specific part. In this case, the user cannot treat the specific part unless the reduction in incision capability is not canceled, and the procedure (treatment) unnecessarily takes time.

According to the fourth embodiment, a reduction in incision capability (i.e., the control process performed on the treatment tool 210 corresponding to the degree of closeness) can be canceled based on at least one of an input from the user and the elapsed time. This makes it possible to improve the safety of the procedure by reducing the incision capability, and prevent a situation in which the procedure (treatment) time unnecessarily increases by canceling a reduction in incision capability.

### 8. Fifth Embodiment

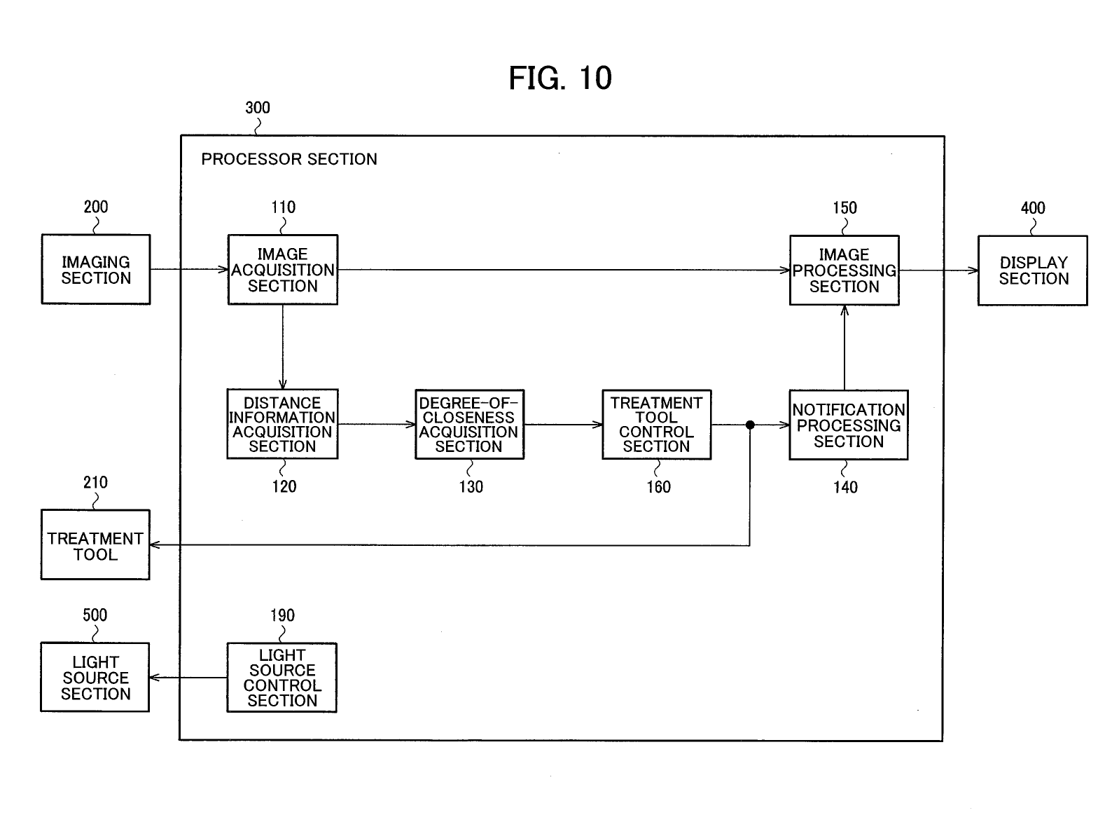

FIG. 10 illustrates a configuration example of an endoscope apparatus according to a fifth embodiment of the invention. The endoscope apparatus includes an imaging section 200, a treatment tool 210, a processor section 300, a display section 400, and a light source section 500. The processor section 300 includes an image acquisition section 110, a distance information acquisition section 120, a degree-of-closeness acquisition section 130, a notification processing section 140, an image processing section 150, a treatment tool control section 160, and a light source control section 190. Note that the same elements as those described above in connection with the above embodiments are respectively indicated by the same reference signs, and description thereof is appropriately omitted.

In the fifth embodiment, an image captured using special light that can capture a deep blood vessel is acquired. A deep blood vessel is detected from the captured image, and distance information about the distance from the treatment tool to the deep blood vessel is acquired using the depth of the deep blood vessel from the object.

Specifically, the light source section 500 generates normal light (white light) that has a white wavelength band, and special light that has a specific wavelength band as illumination light. For example, the light source section 500 includes a light source that generates the normal light, and a filter that allows the special light to pass through. The light source section 500 emits the normal light when the filter is not inserted into the optical path, and emits the special light when the filter is inserted into the optical path. Alternatively, the light source section 500 may include a light source that generates the normal light, and a light source that generates the special light, and emit the normal light or the special light by switching the light source.

The light source control section 190 controls the light source section 500, and causes the light source section 500 to selectively emit the normal light or the special light as illumination light. The normal light and the special light may be switched based on an instruction issued by the user, or the normal light and the special light may be automatically and alternately emitted, for example.

The specific wavelength band is a band that is narrower than the wavelength band (e.g., 380 to 650 nm) of white light (i.e., narrow-band imaging (NBI)), and is the wavelength band of light absorbed by hemoglobin in blood. More specifically, the wavelength band of light absorbed by hemoglobin is 390 to 445 nm (B2 component or first narrow-band light) or 530 to 550 nm (G2 component or second narrow-band light). A wavelength band of 390 to 445 nm or 530 to 550 nm is selected from the viewpoint of absorption by hemoglobin and the ability to reach a surface area or a deep area of tissue. Note that the wavelength band is not limited thereto. For example, the lower limit of the wavelength band may decrease by about 0 to 10%, and the upper limit of the wavelength band may increase by about 0 to 10%, depending on a variation factor (e.g., experimental results for absorption by hemoglobin and the ability to reach a surface area or a deep area of tissue).

The imaging section 200 captures the special light using a normal RGB image sensor, for example. The image acquisition section 110 acquires the B component (i.e., B2 component) of the image input from the imaging section 200, and the G component (i.e., G2 component) of the image input from the imaging section 200 as a special light image. Since a deep blood vessel present in an area deeper than the surface of tissue can be captured using the B2 component and the G2 component of the special light, the special light image includes a deep blood vessel captured using the B2 component, and a deep blood vessel captured using the G2 component. Since the B2 component and the G2 component differ in reachable depth from the surface of tissue, the deep blood vessel captured using the B2 component and the deep blood vessel captured using the G2 component differ in depth.

The distance information acquisition section 120 detects the blood vessel from the image of the B2 component and the image of the G2 component. Since the depth of a blood vessel that can be captured using each component is known in advance, the distance information acquisition section 120 acquires the depth of the blood vessel as the distance information about the blood vessel in the depth direction. The distance information acquisition section 120 calculates the three-dimensional distance from the treatment tool to the blood vessel based on the position of the treatment tool and the position of the blood vessel in the image plane, and the depth of the blood vessel captured using each component. The degree-of-closeness acquisition section 130 converts the three-dimensional distance into the degree of closeness is the same manner as described above in connection with the first embodiment.

In the fifth embodiment, the special light image may be acquired as described below. Specifically, the image acquisition section 110 may acquire the special light image by inputting the G component (i.e., G2 component) of the image input from the imaging section 200 to the R channel, and inputting the B component (i.e., B2 component) of the image input from the imaging section 200 to the G channel and the B channel. A lesion area (e.g., epidermoid cancer) that cannot be easily observed using the normal light can be displayed in brown or the like using such a special light image, and it is possible to prevent a situation in which the lesion area is missed.

The imaging device and the like according to the embodiments of the invention may include a processor and a memory. The processor may be a central processing unit (CPU), for example. Note that the processor is not limited to a CPU. Various types of processors such as a graphics processing unit (GPU) and a digital signal processor (DSP) may also be used. The processor may be a hardware circuit such as an application specific integrated circuit (ASIC). The memory stores a computer-readable instruction. Each section of the imaging device and the like according to the embodiments of the invention is implemented by causing the processor to execute the instruction. The memory may be a semiconductor memory (e.g., SRAM or DRAM), a register, a hard disk, or the like. The instruction may be an instruction included in an instruction set of a program, or may be an instruction that causes a hardware circuit of the processor to operate.

The embodiments according to the invention and the modifications thereof have been described above. Note that the invention is not limited to the above embodiments and the modifications thereof. Various modifications and variations may be made of the above embodiments and the modifications thereof without departing from the scope of the invention. A plurality of elements described in connection with the above embodiments and the modifications thereof may be appropriately combined to implement various configurations. For example, some of the elements described in connection with the above embodiments and the modifications thereof may be omitted. Some of the elements described above in connection with different embodiments or modifications thereof may be appropriately combined. Specifically, various modifications and applications are possible without materially departing from the novel teachings and advantages of the invention. Any term cited with a different term having a broader meaning or the same meaning at least once in the specification and the drawings can be replaced by the different term in any place in the specification and the drawings.

What is claimed is:

1. An endoscope apparatus comprising:
a distance information acquisition section that detects a position of a treatment tool and a position of a specific part, and acquires distance information about a distance between the specific part and the treatment tool based on the detected position of the treatment tool and the detected position of the specific part;
a degree-of-closeness acquisition section that acquires a degree of closeness between the specific part and the treatment tool based on the distance information; and
a treatment tool control section that controls at least one of an incision-related setting and a bleeding arrest-related setting of the treatment tool based on the degree of closeness.

2. The endoscope apparatus as defined in claim 1, further comprising:
a specific part information acquisition section that acquires specific part information, the specific part information being attribute information about the specific part,
the treatment tool control section controlling at least one of the incision-related setting and the bleeding arrest-related setting of the treatment tool based on the degree of closeness and the specific part information.

3. The endoscope apparatus as defined in claim 2,
the specific part information being thickness information about the specific part, and
the treatment tool control section reducing an incision capability of the treatment tool when the degree of closeness is larger than a threshold value, and increasing a degree of reduction in the incision capability as a thickness of the specific part increases.

4. The endoscope apparatus as defined in claim 3,
the specific part being a blood vessel, and
the thickness information being a diameter of the blood vessel.

5. The endoscope apparatus as defined in claim 2,
the specific part information being information that indicates a type of the specific part, and
the treatment tool control section reducing an incision capability of the treatment tool as the degree of closeness increases when the degree of closeness is larger than a threshold value provided that the specific part is a blood vessel, and setting the incision capability to be equal to or less than a given level when the degree of closeness is larger than the threshold value provided that the specific part is a nerve.

6. The endoscope apparatus as defined in claim 1, further comprising:
a treatment tool information acquisition section that acquires treatment tool information, the treatment tool information being attribute information about the treatment tool,
the treatment tool control section controlling at least one of the incision-related setting and the bleeding arrest-related setting of the treatment tool based on the degree of closeness and the treatment tool information.

7. The endoscope apparatus as defined in claim 6,
the treatment tool control section reducing an incision capability of the treatment tool when the degree of closeness is larger than a threshold value, and changing the threshold value corresponding to the treatment tool information.

8. The endoscope apparatus as defined in claim 7,
the specific part being a blood vessel,
the treatment tool information being a bleeding arrest capability of the treatment tool, and
the treatment tool control section decreasing the threshold value as the bleeding arrest capability decreases.

9. The endoscope apparatus as defined in claim 7,
the treatment tool information being information that indicates a type of the treatment tool, and
the treatment tool control section setting the threshold value to a first threshold value when the treatment tool is an electrosurgical knife, and setting the threshold value to a second threshold value when the treatment tool is an ultrasonic surgical knife, the second threshold value being smaller than the first threshold value.

10. The endoscope apparatus as defined in claim 1,
the treatment tool control section reducing an incision capability of the treatment tool when the degree of closeness is larger than a threshold value.

11. The endoscope apparatus as defined in claim 1, further comprising:
a control cancellation section that cancels a control process performed by the treatment tool control section based on at least one of an input from a user and an elapsed time.

12. The endoscope apparatus as defined in claim 11,
the treatment tool control section performing a control process that reduces an incision capability of the treatment tool when the degree of closeness is larger than a threshold value, and
the control cancellation section canceling the control process that reduces the incision capability when the user has performed an input, or when the degree of closeness has not changed by a given amount within a given time, or when the user has performed an input, and the degree of closeness has not changed by the given amount within the given time.

13. The endoscope apparatus as defined in claim 1, further comprising:
a notification processing section that notifies a user of a control state implemented by the treatment tool control section.

14. The endoscope apparatus as defined in claim 13,
the treatment tool control section reducing an incision capability of the treatment tool when the degree of closeness is larger than a threshold value, and
the notification processing section notifying the user that the incision capability is reduced.

15. The endoscope apparatus as defined in claim 1, further comprising:
an image acquisition section that acquires a captured image captured by an imaging section, the captured image including an image of the specific part and the treatment tool,
the distance information acquisition section detecting the position of the treatment tool and the position of the specific part from the captured image, and acquiring the distance information about the distance from the treatment tool to the specific part based on the detected position of the treatment tool and the detected position of the specific part.

16. The endoscope apparatus as defined in claim 1,
the specific part being a linear or tubular part.

17. The endoscope apparatus as defined in claim 16,
the linear or tubular part being a blood vessel, a nerve, or a urinary duct.

18. A method for operating an endoscope apparatus comprising:
detecting a position of a treatment tool and a position of a specific part, and acquiring distance information about a distance between the specific part and the treatment tool based on the detected position of the treatment tool and the detected position of the specific part;
acquiring a degree of closeness between the specific part and the treatment tool based on the distance information; and
controlling at least one of an incision-related setting and a bleeding arrest-related setting of the treatment tool based on the degree of closeness.
